(19) United States
(12) Patent Application Publication
(10) Pub. No.: US 2024/0122953 A1
(43) Pub. Date: Apr. 18, 2024

(54) METHODS FOR REDUCING PATHOGENIC E COLI BY SELECTIVE FEED ADDITIVE INTERVENTION

(71) Applicant: DSM IP Assets B.V., Heerlen (NL)

(72) Inventors: Ghislain SCHYNS, Kaiseraugst (CH); Joshua CLAYPOOL, Kaiseraugst (CH); Kevin FREEMAN, Kaiseraugst (CH)

(21) Appl. No.: 18/546,264

(22) PCT Filed: Feb. 15, 2022
(86) PCT No.: PCT/EP2022/053678

§ 371 Date: Aug. 11, 2023

Related U.S. Application Data
(60) Provisional application No. 63/149,812, filed on Feb. 16, 2021.

(51) Int. Cl. A61K 31/702; A23K 20/163; A23K 50/75; A61P 31/04; C12Q 1/689

(52) U.S. Cl. CPC A61K 31/702; A23K 20/163; A23K 50/75; A61P 31/04; C12Q 1/689; C12Q 2600/156

(57) ABSTRACT

The present disclosure relates to methods of modulating level of pathogenic E. coli (EHEC, EPEC, APEC) present in the gastrointestinal tract of an animal by administering saccharide compositions comprising an anhydro moiety. The presence/load with pathogenic E. coli strains can be assessed via the level of LEE and non-LEE pathogenic genes in the microbiome of the host animal.

### CROSS-REFERENCE TO RELATED APPLICATIONS

This application is the U.S. national phase of International Application No. PCT/EP2022/053678 filed Feb. 15, 2022 which designated the U.S. and claims priority to U.S. 63/149,812 filed Feb. 16, 2021, the entire contents of each of which are hereby incorporated by reference.

### TECHNICAL FIELD

The invention pertains to the reduction of E. coli pathogenesis. The invention pertains to a method for improving the health of production animals by reducing the population of E. coli pathogens in the gastrointestinal tract of the animals. More specifically, the invention pertains to methods for reducing the population of locus for enterocyte effacement (LEE) genes and the non-LEE pathogenic genes of pathogenic E. coli strains in the microbiome of their host animal. The invention also pertains to the reduction of the population of Bacteroides thetaiotaomicron in the microbiome of their host animal.

### BACKGROUND INFORMATION

Escherichia coli is an extremely versatile microorganism. In addition to being a member of the normal intestinal flora, strains of E. coli also cause bladder infections, meningitis, and diarrhea. Diarrheagenic E. coli include at least five types of E. coli, which cause various symptoms ranging from cholera-like diarrhea to extreme colitis. Each type of diarrheagenic E. coli possesses a particular set of virulence factors, including adhesins, invasins, and/or toxins, which are responsible for causing a specific type of diarrhea.

Enteropathogenic E. coli (EPEC), is a predominant cause of infantile diarrhea worldwide. EPEC disease is characterized by watery diarrhea of varying severity, with vomiting and fever often accompanying the fluid loss. In addition to isolated outbreaks in daycares and nurseries in developed countries, EPEC poses a major endemic health threat to young children (<6 months) in developing countries.

Enterohemorragic E. coli(EHEC), also called Shiga toxin producing E. coli(STEC), causes a more severe diarrhea than EPEC (enteric colitis) and in approximately 10% of cases, this disease progresses to an often fatal kidney disease, hemolytic uremic syndrome (HUS). EHEC O157:H7 is the most common serotype in Canada and the United States, and is associated with food and water poisoning (Perna et al., 2001, Nature 409: 529-533). Other serotypes of EHEC also cause significant problems worldwide. EHEC colonizes cattle and causes A/E lesions, but does not cause disease in adult animals, and instead sheds organisms into the environment. This however causes serious health problems as a relatively few EHEC are necessary to infect humans.

Unlike other E. coli diarrheas, such as enterotoxigenic E. coli, diarrhea caused by EHEC and EPEC is not mediated by a toxin. Instead, EPEC and EHEC bind to intestinal surfaces (EPEC the small bowel, EHEC the large bowel) and cause a characteristic histological lesion, called the attaching and effacing (A/E) lesion (Tauschek, et. al. 2002, Mol. Microbiol 44; 1533-1550.). A/E lesions are marked by dissolution of the intestinal brush border surface and loss of epithelial microvilli (effacement) at the sites of bacterial attachment. Once bound, bacteria reside upon cup-like projections or pedestals. Underlying this pedestal in the epithelial cell are several cytoskeletal components, including actin and actin associated cytoskeletal proteins. Formation of A/E lesions and actin-rich pedestals beneath attaching bacteria is the histopathological hallmark of A/E pathogens (Nataro, et. al., 1998, Clin Microbiol Rev 11: 142-201, and Frankel et al., 1998 Mol Microbiol 30: 911-921).

EPEC and EHEC belong to a family of A/E pathogens, including several EPEC-like animal pathogens that cause disease in rabbits (REPEC), pigs (PEPEC), and mice (Citrobacter rodentium). These pathogens contain pathogenicity islands (PAls) that encode specialized secretion systems and secreted virulence factors critical for disease. The genes required for the formation of A/E lesions are thought to be clustered together in a single chromosomal pathogenicity island known as the locus for enterocyte effacement (LEE), which includes regulatory elements, a type Ill secretion system (TTSS), secreted effector proteins, and their cognate chaperones (Elliott et al., 1998, Mol Microbiol 28: 1-4. Perna, et al., 1998, Infect Immun 66: 3810-3817. Zhu, et al., 2001, Infect Immun 69: 2107-2115; Deng et al., 2001, Infect Immun 69: 6323-6335.

The LEE contains 41 genes, making it one of the more complex PAls. The main function of the LEE TTSS is to deliver effectors into host tells, where they subvert host cell functions and mediate disease. Five LEE-encoded effectors (Tir, EspG, EspF, Map, and EspH) have been identified. Tir (for translocated intimin receptor) is translocated into host cells where it binds host cytoskeletal and signaling proteins and initiates actin polymerization at the site of bacterial attachment, resulting in formation of actin pedestal structures underneath adherent bacteria, which directly interact with the extracellular loop of Tir via the bacterial outer membrane protein intimin. CesT plays a role as a chaperone for Tir stability and secretion.

Four other LEE-encoded TTSS-translocated effectors have been characterized in A/E pathogens: EspH enhances elongation of actin pedestals; EspF plays a role in disassembly of tight junctions between intestinal epithelial cells; EspG is related to the Shigella microtubule-binding effector VirA; and Map localizes to mitochondria, but also has a role in actin dynamics. Ler (for LEE encoded regulator) is the only LEE encoded regulator identified.

Avian Pathogenic E. coli(APEC), the etiological agent of extra-intestinal infections in birds, is a pathotype that belongs to the ExPEC group. Extraintestinal infections caused by APEC are known as colibacillosis and characterized by fibrinous lesions around visceral organs, such as septicaemia, enteritis, granulomas, omphalitis, sinusitis, airsacculitis, arthritis/synovitis, peritonitis, pericarditis, perihepatitis, cellulitis, and swollen head syndrome (Kunert et al., 2015, World's Poultry Science Journal. 71; 249-258). APEC infections also lead to reduced yield, quality, and hatching of eggs. The potential for zoonotic transmission must be considered, since poultry serves as the main host for APEC and the consumption of undercooked poultry may infect humans, which can serve as a reservoir of this pathotype (Markland et al., Zoonoses and Public Health. 2015; doi: 10.1111/zph.12194).

This pathotype is the etiologic agent of extra-intestinal infections in broiler chickens and laying hens, and these are collectively known as colibacillosis. Colibacillosis is responsible for significant economic losses in many countries. It affects all cycles of production and all sectors of the poultry industry. It causes high morbidity and mortality in broiler chickens and laying hens. An E. coli strain can be designated as APEC when isolated from birds with characteristics of colibacillosis lesions and birds that were killed by this bacterium. E. coli designated as APEC must possess some virulence genes such as encoding adhesins, iron-scavenging systems, protectins, and other virulence traits. Control methods based only on predisposing factors were not effective in preventing colibacillosis.

The bacterium Bacteroides thetaiotaomicron is one of the most abundant species of the phylum Bacteroidetes, in both humans and mice, Bacteroidetes being one of the three major phyla of the intestinal microflora (Qin J et al., 2010, Nature, 464, pp. 59-65). It has been observed that, the expression of EHEC NIPH-11060424 genes involved in metabolism, colonization and virulence is modulated in response to direct contact with B. thetaiotaomicron and to soluble factors released from B. thetaiotaomicron. It was suggested that direct contact with B. thetaiotaomicron could function as a niche specific signal that primes EHEC for a more efficient interaction with the host cells thus increasing virulence potential (Iversen et al., 2015, PLoS ONE 10(2): e0118140. doi:10.1371/journal. pone.0118140).

Traditionally, reduction or elimination of pathogenic E coli strains in production animals were often focusing on bacteria supersession in the gastrointestinal tract of the animal by means of pharmaceuticals such as antibiotics. Given the increasing knowledge about microbiome and their role in the digestive system of the host animals, there is a need to identify novel, non-antibiotics ways of reducing pathogenic E. coli populations in production animals. In other words, there is a need to identify novel ways of modulating the pathogenic E. coli population in the microbiome and thus improve the health of the host animal.

### SUMMARY OF THE INVENTION

The present invention is directed to a method for reducing the population of exogenous locus for enterocyte effacement (LEE) genes and exogenous non-LEE pathogenic genes of Enterohemorrhagic E. coli (EHEC), Enteropathogenic E. coli (EPEC), and Avian Pathogenic E. coli (APEC) in the gastrointestinal tract (GIT) of an animal, comprising feeding said animal with one of more of the following feed additives: oligosaccharides, and essential oils, wherein the population of exogenous LEE genes and non-LEE pathogenic genes is reduced by at least 10% lower than that of a control animal which is fed with the same diet except for said feed additives. In one embodiment, the population of exogenous LEE genes and non-LEE pathogenic genes is measured as % ratio of the combined copy numbers of LEE genes and non-LEE genes detected within the microbiome of said animal vs. the total copy number of genes detected within said microbiome.

The present invention is also directed to a method for reducing the population of Bacteroides thetaiotaomicron in the gastrointestinal tract (GIT) of an animal, comprising feeding said animal with one of more of the following feed additives: oligosaccharides, and essential oils, wherein the population of Bacteroides thetaiotaomicron is reduced by at least 10% lower than that of a control animal which is fed with the same diet except for said feed additives. In one embodiment, the population of Bacteroides thetaiotaomicron is measured as % ratio of the population of Bacteroides thetaiotaomicron detected within the microbiome of said animal against the total population of microbes within said microbiome.

The present invention is further directed to a method for reducing systemic inflammation and/or local inflammation of an animal caused by E. coli infection, comprising feeding said animal with one of more of the following feed additives: oligosaccharides, and essential oils, wherein the systemic inflammation and/or local inflammation of the animal is reduced by at least 10% lower than that of a control animal which are fed with the same diet except for said feed additives. In one embodiment, the reduction of inflammation is measured as % ratio of the copy number of LEE and non-LEE genes detected within the microbiome of said animal against the total copy number of genes detected within said microbiome.

In some embodiments of the above inventions, the microbiome is collected from either a fecal sample of the animal or a sample collected within the GIT of the animal. In some embodiments, the gene copy number measurement is performed by RT-PCR counting, full length 16S RNA sequencing, or Metagenomic DNA sequencing. In one embodiment, the LEE genes comprise: Tir, Map, EspB, EspF, EspG, EspH, and EspZ, and the non-LEE pathogenic genes comprise: EspG2, EspJ, EspM1/2, EspT, EspW, Cif, NleA, NIeB, NleC, NIeD, NleE, NIeF, and NleH. In one embodiment, the method of the present invention is applicable to production animal.

### BRIEF DESCRIPTION OF DRAWINGS

FIG. 1 is a graph showing the change of relative abundance of B. thetaiotamicron in the GIT of chicken that were fed a diet comprising an oligosaccharide preparation of the invention and that of the control chicken group that were fed a control diet not comprising the oligosaccharide preparation; the latter was set to 100%.

FIG. 2 is a graph showing the relative abundance of the relative abundance of LEE genes and non-LEE pathogenic genes in the metagenome of the GIT of chicken that were fed a diet comprising an oligosaccharide preparation of the invention and that of the control chicken group that were fed a control diet not comprising the oligosaccharide preparation.

### DETAILED DESCRIPTION OF THE INVENTION

The terminology used herein is for the purpose of describing particular cases only and is not intended to be limiting. As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof are used in either the detailed description and/or the claims, such terms are intended to be inclusive in a manner similar to the term “comprising.”

It is understood that terms such as “comprises,” “comprised,” “comprising,” and the like have the meaning attributed to it in U.S. Patent law; i.e., they mean “includes,” “included,” “including,” and the like and are intended to be inclusive or open ended and does not exclude additional, unrecited elements or method steps; and that terms such as “consisting essentially of” and “consists essentially of” have the meaning ascribed to them in U.S. Patent law; i.e., they allow for elements not explicitly recited, but exclude elements that are found in the prior art or that affect a basic or novel characteristic of the invention.

### Definitions

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or B; A or C; B or C; A and B; A and C; B and C; A (alone); B (alone); and C (alone).

The term “oligosaccharide” may refer to a monosaccharide or a compound containing two or more monosaccharide subunits linked by glycosidic bonds. An oligosaccharide in an oligosaccharide preparation may also refer to an anhydro-monosaccharide or a compound containing two or more monosaccharide subunits, where at least one monosaccharide unit is replaced by an anhydro-subunit.

An “oligosaccharide” may be optionally functionalized. As used herein, the term “oligosaccharide” encompasses all species of the oligosaccharide, wherein each of the monosaccharide subunit in the oligosaccharide is independently and optionally functionalized and/or replaced with its corresponding anhydro-monosaccharide subunit.

The terms “oligosaccharide preparation” and “synthetic oligosaccharide preparation” are used interchangeably herein and refer to an oligosaccharide preparation that was manufactured as described in detail in WO 2020/097458, and as described below.

An “anhydro-subunit” may be a product of reversible thermal dehydration of a monosaccharide (or monosaccharide subunit) or a sugar caramelization product. For example, an “anhydro-subunit” may be an anhydro-monosaccharide such as anhydro-glucose. As another example, an “anhydro-subunit” may be linked with one or more regular or anhydro-monosaccharide subunits via glycosidic linkage.

An oligosaccharide in an oligosaccharide preparation may be characterized to contain two or more monosaccharide subunits linked by glycosidic bonds. In this regard, a “gluco-oligosaccharide” may refer to a glucose or a compound containing two or more glucose monosaccharide subunits linked by glycosidic bonds. A “gluco-oligosaccharide” may also refer to an anhydro-glucose or a compound containing two or more glucose monosaccharide subunits linked by glycosidic bonds, wherein at least one monosaccharide subunit is replaced with an anhydro-glucose subunit. Similarly, a “galacto-oligosaccharide” may refer to a galactose or a compound containing two or more galactose monosaccharide subunits linked by glycosidic bonds. A “galacto-oligosaccharide” may also refer to an anhydro-galactose or a compound containing two or more galactose monosaccharide subunits linked by glycosidic bonds, wherein at least one monosaccharide subunit is replaced with an anhydro-galactose subunit. Analogously, a gluco-galactose-oligosaccharide may be a gluco-oligosaccharide, a galacto-oligosaccharide, or a compound containing one or more glucose monosaccharide subunits and one or more galactose monosaccharide subunits linked by glycosidic bonds, wherein at least one of the monosaccharide subunits is replaced with its respective anhydro-monosaccharide subunit.

A gluco-galacto-xylo-oligosaccharide may refer to a compound produced by the condensation reaction of glucose, galactose, and xylose. An oligosaccharide preparation comprising gluco-galacto-xylo-oligosaccharides may comprise gluco-galactose-oligosaccharides, gluco-xylo-oligosaccharides, galacto-xylo-oligosaccharides, and compounds containing one or more glucose monosaccharide subunits, one or more xylose monosaccharide subunits, and one or more galactose monosaccharide subunits linked by glycosidic bonds.

As used herein, the term “monosaccharide unit” and “monosaccharide subunit” may be used interchangeably, unless suggested otherwise. A “monosaccharide subunit” may refer to a monosaccharide monomer in an oligosaccharide. For an oligosaccharide in an oligosaccharide preparation having a degree of polymerization of 1, the oligosaccharide may be referred to as a monosaccharide subunit or monosaccharide. For an oligosaccharide in an oligosaccharide preparation having a degree of polymerization higher than 1, its monosaccharide subunits are linked via glycosidic bonds.

As used herein, the term “regular monosaccharide” may refer to a monosaccharide that does not contain an anhydro-subunit. The term “regular disaccharide” may refer to a disaccharide that does not contain an anhydro-subunit. Accordingly, the term “regular subunit” may refer to a subunit that is not an anhydro-subunit.

The term “relative abundance” or “abundance,” as used herein, may refer to the abundance of a species in terms of how common or rare the species exists. For example, a DP1 fraction comprising 10% anhydro-subunit containing oligosaccharides by relative abundance may refer to a plurality of DP1 oligosaccharides, wherein 10%, by number, of the DP1 oligosaccharides are anhydro-monosaccharides.

Degree of Polymerization (DP) Distribution: A distribution of the degree of polymerization of the oligosaccharide preparation may be determined by any suitable analytical method and instrumentation, including but not limited to end group method, osmotic pressure (osmometry), ultracentrifugation, viscosity measurements, light scattering method, size exclusion chromatography (SEC), SEC-MALLS, field flow fractionation (FFF), asymmetric flow field flow fractionation (A4F), high-performance liquid chromatography (HPLC), and mass spectrometry (MS). For example, the distribution of the degree of polymerization may be determined and/or detected by mass spectrometry, such as MALDI-MS, LC-MS, or GC-MS. For another example, the distribution of the degree of polymerization may be determined and/or detected by SEC, such as gel permeation chromatography (GPC). As yet another example, the distribution of the degree of polymerization may be determined and/or detected by HPLC, FFF, or A4F. In another example, the degree of polymerization of the oligosaccharide preparation may be determined based on its molecular weight and molecular weight distribution (for a more detailed description see WO 2020/097458).

Anhydro-subunit Level: In some embodiments, each of the n fractions of oligosaccharides of the oligosaccharide preparation as described herein independently comprises an anhydro-subunit level. For instance, in some embodiments, the DP1 fraction comprises 10% anhydro-subunit containing oligosaccharides by relative abundance, and the DP2 fraction comprises 15% anhydro-subunit containing oligosaccharides by relative abundance. For another example, in some embodiments, DP1, DP2, and DP3 fraction each comprises 5%, 10%, and 2% anhydro-subunit containing oligosaccharides by relative abundance, respectively. In other embodiments, two or more fractions of oligosaccharides may comprise similar level of anhydro-subunit containing oligosaccharides. For example, in some embodiments, the DP1 and DP3 fraction each comprises about 5% anhydro-subunit containing oligosaccharides by relative abundance.

The level of anhydro-subunits may be determined by any suitable analytical methods, such as nuclear magnetic resonance (NMR) spectroscopy, mass spectrometry, HPLC, FFF, A4F, or any combination thereof. In some embodiments, the level of anhydro-subunits is determined, at least in part, by mass spectrometry such as MALDI-MS. In some embodiments, the level of anhydro-subunits may be determined, at least in part, by NMR. In some embodiments, the level of anhydro-subunits may be determined, at least in part, by HPLC. For example, in some embodiments, the level of anhydro-subunits may be determined by MALDI-MS, as illustrated in more detail in WO 2020/097458.

Glycosidic Linkages: In some embodiments, the oligosaccharide preparation described herein comprise a variety of glycosidic linkages. The type and distribution of the glycosidic linkages may depend on the source and manufacturing method of the oligosaccharide preparation. In some embodiments, the type and distribution of various glycosidic linkages may be determined and/or detected by any suitable methods known in the art such as NMR. For example, in some embodiments, the glycosidic linkages are determined and/or detected by proton NMR, carbon NMR, 2D NMR such as 2D JRES, HSQC, HMBC, DOSY, COSY, ECOSY, TOCSY, NOESY, or ROESY, or any combination thereof. In some embodiments, the glycosidic linkages are determined and/or detected, at least in part, by proton NMR. In some embodiments, the glycosidic linkages are determined and/or detected, at least in part, by carbon NMR. In some embodiments, the glycosidic linkages are determined and/or detected, at least in part, by 2D HSQC NMR.

In some embodiments, an oligosaccharide preparation may comprise one or more α-(1,2) glycosidic linkages, α-(1,3) glycosidic linkages, α-(1,4) glycosidic linkages, α-(1,6) glycosidic linkages, β-(1,2) glycosidic linkages, β-(1,3) glycosidic linkages, β-(1,4) glycosidic linkages, β-(1,6) glycosidic linkages, α(1, 1)α glycosidic linkages, α(1, 1)β glycosidic linkages, β(1, 1)β glycosidic linkages, or any combination thereof.

In some embodiments, the oligosaccharide preparations have a glycosidic bond type distribution of about from 0 to 60 mol %, 5 to 55 mol %, 5 to 50 mol %, 5 to 45 mol %, 5 to 40 mol %, 5 to 35 mol %, 5 to 30 mol %, 5 to 25 mol %, 10 to 60 mol %, 10 to 55 mol %, 10 to 50 mol %, 10 to 45 mol %, 10 to 40 mol %, 10 to 35 mol %, 15 to 60 mol %, 15 to 55 mol %, 15 to 50 mol %, 15 to 45 mol %, 15 to 40 mol %, 15 to 35 mol %, 20 to 60 mol %, 20 to 55 mol %, 20 to 50 mol %, 20 to 45 mol %, 20 to 40 mol %, 20 to 35 mol %, 25 to 60 mol %, 25 to 55 mol %, 25 to 50 mol %, 25 to 45 mol %, 25 to 40 mol %, or 25 to 35 mol % of α-(1,6) glycosidic linkages.

Molecular Weight: The molecular weight and molecular weight distribution of the oligosaccharide preparation may be determined by any suitable analytical means and instrumentation, such as end group method, osmotic pressure (osmometry), ultracentrifugation, viscosity measurements, light scattering method, SEC, SEC-MALLS, FFF, A4F, HPLC, and mass spectrometry. In some embodiments, the molecular weight and molecular weight distribution are determined by mass spectrometry, such as MALDI-MS, LC-MS, or GC-MS. In some embodiments, the molecular weight and molecular weight distribution are determined by size exclusion chromatography (SEC), such as gel permeation chromatography (GPC). In other embodiments, the molecular weight and molecular weight distribution are determined by HPLC. In some embodiments, the molecular weight and molecular weight distribution are determined by MALDI-MS.

In some embodiments, the weight average molecular weight of the oligosaccharide preparation is about from 100 to 10000 g/mol, 200 to 8000 g/mol, 300 to 5000 g/mol, 500 to 5000 g/mol, 700 to 5000 g/mol, 900 to 5000 g/mol, 1100 to 5000 g/mol, 1300 to 5000 g/mol, 1500 to 5000 g/mol, 1700 to 5000 g/mol, 300 to 4500 g/mol, 500 to 4500 g/mol, 700 to 4500 g/mol, 900 to 4500 g/mol, 1100 to 4500 g/mol, 1300 to 4500 g/mol, 1500 to 4500 g/mol, 1700 to 4500 g/mol, 1900 to 4500 g/mol, 300 to 4000 g/mol, 500 to 4000 g/mol, 700 to 4000 g/mol, 900 to 4000 g/mol, 1100 to 4000 g/mol, 1300 to 4000 g/mol, 1500 to 4000 g/mol, 1700 to 4000 g/mol, 1900 to 4000 g/mol, 300 to 3000 g/mol, 500 to 3000 g/mol, 700 to 3000 g/mol, 900 to 3000 g/mol, 1100 to 3000 g/mol, 1300 to 3000 g/mol, 1500 to 3000 g/mol, 1700 to 3000 g/mol, 1900 to 3000 g/mol, 2100 to 3000 g/mol, 300 to 2500 g/mol, 500 to 2500 g/mol, 700 to 2500 g/mol, 900 to 2500 g/mol, 1100 to 2500 g/mol, 1300 to 2500 g/mol, 1500 to 2500 g/mol, 1700 to 2500 g/mol, 1900 to 2500 g/mol, 2100 to 2500 g/mol, 300 to 1500 g/mol, 500 to 1500 g/mol, 700 to 1500 g/mol, 900 to 1500 g/mol, 1100 to 1500 g/mol, 1300 to 1500 g/mol, 2000-2800 g/mol, 2100-2700 g/mol, 2200-2600 g/mol, 2300-2500 g/mol, or 2320-2420 g/mol. In some embodiments, the weight average molecular weight of the oligosaccharide preparation is about from 2000 to 2800 g/mol, 2100 to 2700 g/mol, 2200 to 2600 g/mol, 2300 to 2500 g/mol, or 2320 to 2420 g/mol.

Types of Oligosaccharides: In some embodiments, the species of oligosaccharides present in an oligosaccharide preparation referred to herein may depend on the type of the one or more feed sugars. For example, in some embodiments, the oligosaccharide preparations comprise a gluco-oligosaccharide when the feed sugars comprise glucose. For example, in some embodiments, the oligosaccharide preparations comprise a galacto-oligosaccharide when the feed sugars comprise galactose. For another example, in some embodiments, the oligosaccharide preparations comprise gluco-galacto-oligosaccharides when the feed sugars comprise galactose and glucose.

In some embodiments, the oligosaccharide preparations comprise one or more species of monosaccharide subunits. In some embodiments, the oligosaccharide preparation may comprise oligosaccharides with 1, 2, 3, 4, 5, 6, 7, 8, 9, 10, 11, 12, 13, 14, 15, 16, 17, 18, 19, 20, or more different species of monosaccharides subunits.

Method of Manufacturing Oliqosaccharide Preparations: The Method of manufacturing an oligosaccharide preparation according to the invention is described in detail in WO 2020/097458, comprising heating an aqueous composition comprising one or more feed sugars and a catalyst to a temperature and for a time sufficient to induce polymerization, wherein the catalyst is selected from the group consisting of: (+)-camphor-10-sulfonic acid; 2-pyridinesulfonic acid; 3-pyridinesulfonic acid; 8-hydroxy-5-quinolinesulfonic acid hydrate; a-hydroxy-2-pyridinemethanesulfonic acid; (β)-camphor-10-sulfonic acid; butylphosphonic acid; diphenylphosphinic acid; hexylphosphonic acid; methylphosphonic acid; phenylphosphinic acid; phenylphosphonic acid; tert-butylphosphonic acid; SS)-VAPOL hydrogenphosphate; 6-quinolinesulfonic acid, 3-(1-pyridinio)-1-propanesulfonate; 2-(2-pyridinyl)ethanesulfonic acid; 3-(2-pyridyl)-5,6-diphenyl-1,2,4-triazine-p,p′-disulfonic acid monosodium salt hydrate; 1,1′-binaphthyl-2,2′-diyl-hydrogenphosphate; bis(4-methoxyphenyl)phosphinic acid; phenyl(3,5-xylyl)phosphinic acid; L-cysteic acid monohydrate; poly(styrene sulfonic acid -co- divinylbenzene); lysine; Ethanedisulfonic acid; Ethanesulfonic acid; Isethionic acid; Homocysteic acid; HEPBS (N-(2-Hydroxyethyl)piperazine-N′-(4-butanesulfonic acid)); HEPES (4-(2-hydroxyethyl)-1-piperazineethanesulfonic acid); 2-Hydroxy-3-morpholinopropanesulfonic acid; 2-(N-morpholino)ethanesulfonic acid; Methanesulfonic acid; Methaniazide; Naphthalene-1-sulfonic acid; Naphthalene-2-sulfonic acid; Perfluorobutanesulfonic acid; 6-sulfoquinovose; Triflic acid; 2-aminoethanesulfonic acid; Benzoic acid; Chloroacetic acid; Trifluoroacetic acid; Caproic acid; Enanthic acid; Caprylic acid; Pelargonic acid; Lauric acid; Pamitic acid; Stearic acid; Arachidic acid; Aspartic acid; Glutamic acid; Serine; Threonine; Glutamine; Cysteine; Glycine; Proline; Alanine; Valine; Isoleucine; Leucine; Methionine; Phenylalanine; Tyrosine; Tryptophan.

In some embodiments, the polymerization of the feed sugars is achieved by a step-growth polymerization. In some embodiments, the polymerization of the feed sugars is achieved by polycondensation.

Feed Sugar: The one or more feed sugars used in the methods of manufacturing oligosaccharide preparations described herein may comprise one or more types of sugars. In some embodiments, the one or more feed sugars comprise monosaccharides, disaccharides, trisaccharides, tetrasaccharides, or any mixtures thereof.

In some embodiments, the one or more feed sugars comprise glucose. In some embodiments, the one or more feed sugars comprise glucose and galactose. In some embodiments, the one or more feed sugars comprise glucose, xylose, and galactose. In some embodiments, the one or more feed sugars comprise glucose and mannose. In some embodiments, the one or more feed sugars comprise glucose and fructose. In some embodiments, the one or more feed sugars comprise glucose, fructose, and galactose. In some embodiments, the one or more feed sugars comprise glucose, galactose, and mannose.

As used herein, the singular forms “a,” “and,” and “the” include plural referents unless the context clearly dictates otherwise. Thus, for example, reference to “an agent” includes a plurality of such agents, and reference to “the oligosaccharide” includes reference to one or more oligosaccharides (or to a plurality of oligosaccharides) and equivalents thereof known to those skilled in the art, and so forth.

When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range, in some instances, will vary between 1% and 15% of the stated number or numerical range.

The terms “microbiome” and “gut microbiome”, which are used interchangeably in this application, refer to microbes such as bacteria, viruses, fungi, mold, protozoa, etc. that reside in the digestive track, and is responsible for converting undigested and unabsorbed components of an animal's diet into thousands of biologically active metabolites. These metabolites interface in turn with the local and systemic physiology of the animal as well as the animal's external environment.

Method of reducing the population of pathogenic E. coli bacteria in the microbiome of an animal

In this invention, a method of improving the health of production animal is shown. A preferred embodiment of the method of the invention relates to a method of improving the health of a production animal by reducing the population of E coli bacteria in the microbiome of the animal. In one embodiment, a method of the invention relates to a method of improving the health of a production animal by reducing the population of pathogenic E coli bacteria in the microbiome of the animal while making lesser or insignificant impact on the non-pathogenic E. coli. In a preferred embodiment, the selective modulation of E. coli population is achieved by reducing the population of exogenous locus for enterocyte effacement (LEE) genes and exogenous non-LEE pathogenic genes of pathogenic E. coli bacteria such as Enterohemorrhagic E. coli (EHEC), Enteropathogenic E. coli (EPEC), and Avian Pathogenic E. coli (APEC) in the microbiome of the animal. In another embodiment, the invention relates to a method of improving the health of a production animal by reducing the population of Bacteroides thetaiotaomicron in the microbiome of the animal. In a specific embodiment, the above health benefit is instigated by feeding the production animal with selective feed additives such as oligosaccharides, and essential oils.

Enterohemorrhagic E. coli (EHEC), Enteropathogenic E. coli (EPEC), and Avian Pathogenic E. coli (APEC) are a diarrheagenic human pathogen. The hallmark of infections by these pathogenic E. coli strains is the formation of the attaching and effacing (A/E) lesion in the intestinal epithelial cells, characterized by the effacement of brush border microvilli and the intimate bacterial attachment to the enterocyte in actin-rich pedestal-like structures. The locus of enterocyte effacement (LEE) in the pathogenic E. coli genome encodes a type Ill protein secretion system (T3SS) that translocates multiple effector proteins into the host cell to subvert cellular functions for the benefit of the pathogen. These effectors are encoded by genes both within and outside the LEE region. In vitro cell culture infections have shown that LEE effectors are required for intimate bacterial attachment to the epithelial cells, whereas non-LEE effectors mostly play a role in modulating inflammation and cell apoptosis in the gut epithelium (Massiel et al., 2020, DOI: 10.5772/intechopen.91677).

Surprisingly, inventors of present application have identified a few selective nutritional feed additives such as oligosaccharides, and essential oils which can significantly interfere with the growth of pathogenic E. coli such as EHEC, EPEC and APEC. It has been shown in the present invention that feeding suitable amount of above selective feed additive can help to reduce the population of LEE genes and non-LEE pathogenic genes in the microbiome of the host animal. In other words, the LEE genes of pathogenic E. coli bacteria, which are responsible for forming the attaching and effacing (A/E) lesion in the gut epithelial cells of the host animal, are reduced in term of its % population within the GIT microbiome of the host animal when treated by selective nutritional additives. Furthermore, it is observed by the inventors of present application that the non-LEE pathogenic genes of pathogenic E. coli bacteria, which are responsible for modulating inflammation and cell apoptosis in the gut epithelium, are also reduced in term of its % population within the GIT microbiome of the host animal. This leads to reduced systemic and local infection of the GIT of the host animal.

It is also observed by the inventors of present application that the selective nutritional feed additives can reduce the % population of E. coli within the GIT microbiome of the host animal.

Specifically, the % population of pathogenic E. coli bacteria within the GIT microbiome is reduced, possibly due to the observed reductions in the abundance of the LEE-encoded and non-LEE-encoded effectors of EHEC, EPEC and APEC.

Equally surprising, inventors of present application found that the same selective nutritional feed additives can significantly reduce the % population of Bacteroides thetaiotaomicron within the GIT microbiome. The virulence of Enterohaemorrhagic E. coli (EHEC) is reported to be coordinated with B. thetaiotaomicron, a gut commensal. Impacting this commensal can have subsequent effects on EHEC. It is known that B. thetaiotaomicron functions as a niche specific signal that primes EHEC for a more efficient interaction with the host cells and thus increasing virulence potential. Thus, reduction or removal of B. thetaiotaomicron from the GIT microbiome can diminish the interaction of EHEC with the GIT epithelial cells of the host animal and thus prevent or alleviate the pathogenicity of E. coli such as EHEC against the host animal.

On the physiology level of the animal, the selective nutritional feed additives identified in this application help to treat diarrhea and nutrient malabsorption and other poor health outcomes of animal. This is achieved by reducing the population of pathogenic E. coli bacteria and/or their coordinator B. thetaiotaomicron in the microbiome of the host animal and thus alleviating the pathogenicity caused by such bacteria.

Thus, a preferred embodiment of the invention relates to a method for reducing the population of exogenous locus for enterocyte effacement (LEE) genes and exogenous non-LEE pathogenic genes of Enterohemorrhagic E. coli(EHEC), Enteropathogenic E. coli(EPEC), and Avian Pathogenic E. coli (APEC) in the gastrointestinal tract (GIT) of an animal, comprising the step of feeding said animal with one of more of the following feed additives: oligosaccharides, and essential oils, wherein the population of exogenous LEE genes and non-LEE pathogenic genes is reduced by at least 10% lower than that of a control animal which is fed with the same diet except for said feed additives.

In one embodiment, the reduction of the population of exogenous LEE genes and non-LEE pathogenic genes is measured by the % ratio of LEE genes and non-LEE genes against the total amount of genes in the microbiome. In other words, the reduction is measured as the change of the population of pathogenic E. coli in the microbiome. In another embodiment, the reduction is measured by the % ratio of LEE genes and non-LEE genes against the copy number of an E. coli housekeeping gene. In other words, the reduction is measured as the change of the population of pathogenic E. coli in the overall E. coli population of the microbiome. In some embodiments, the reduction of the population of the % ratio of LEE genes and non-LEE genes is by at least 5%, at least 15%, at least 20%, at last 30%, at least 40%, at least 50%, at least 60%, at least 70%, at least 80%, or at least 90% lower than that of the control animal. In one embodiment, the LEE genes comprise: Tir, Map, EspB, EspF, EspG, EspH, and EspZ. In another embodiment, the non-LEE pathogenic genes comprise: EspG2, EspJ, EspM1/2, EspT, EspW, Cif, NIeA, NIeB, NIeC, NIeD, NIeE, NIeF, and NIeH.

Another preferred embodiment of the invention relates to a method of reducing the population of Bacteroides thetaiotaomicron in the gastrointestinal tract (GIT) of an animal, comprising feeding said animal with one of more of the following feed additives: oligosaccharides, and essential oils, wherein the population of Bacteroides thetaiotaomicron is reduced by at least 10% lower than that of a control animal which is fed with the same diet except for said feed additives.

In some embodiments, the reduction of the population of the % ratio of Bacteroides thetaiotaomicron against the total number of microbes in the microbiome is by at least 5%, at least 15%, at least 20%, at last 30%, at least 40%, at least 50%, at least 60%, at least 70%, at least 80%, at least 90%, or at least 95% lower than that of the control animal.

Another preferred embodiment of the invention relates to a method of reducing systemic inflammation and/or local inflammation of an animal caused by E. coli infection, comprising feeding said animal with one of more of the following feed additives: oligosaccharides, and essential oils, wherein the systemic inflammation and/or local inflammation of the animal is reduced by at least 10% lower than that of a control animal which are fed with the same diet except for said feed additives.

In some embodiments, the reduction of the population of the % ratio of the inflammation related non-LEE genes against the total amount of genes in the microbiome is by at least 5%, at least 15%, at least 20%, at last 30%, at least 40%, at least 50% at least 55%, or at least 60% lower than that of the control animal. In one embodiment, the inflammation related non-LEE genes comprise: NIeA, NIeB, NIeC, NIeD, NIeE, NIeF, and NIeH.

Another preferred embodiment of the invention relates to a method of reducing the population of E. coli in the gastrointestinal tract (GIT) of an animal, comprising feeding said animal with one of more of the following feed additives: oligosaccharides, and essential oils, wherein the systemic inflammation and/or local inflammation of the animal is reduced by at least 10% lower than that of a control animal which are fed with the same diet except for said feed additives. In a specific embodiment, said E. coli is pathogenic E. coli. In specific embodiment, the pathogenic E. coli comprises EPEC, EHEC, and APEC.

In one embodiment, the population of E. coli in the GIT of the animal is measured as % of copy number of E. coli marker genes within the microbiome of said animal against the total copy number of bacterial marker genes detected within said microbiome. In some embodiments, the reduction of E. coli population in the GIT of the animal is by at least 5%, at least 15%, at least 20%, or at last 30% lower than that of the control animal.

In one embodiment, the microbiome is collected from the fecal digesta sample of the animal. In another embodiment, the microbiome is collected from a location within the GIT of the animal. In an embodiment, the microbiome is collected from the GIT of a chicken. In some embodiments, the location is the duodenum, jejunum, ileum, cecum, or colorectum of a chicken.

Measurement of population of any genes of any microbe in the microbiome or the population of the microbiome can be conducted using any existing or future method which are suitable for the purpose. In one embodiment, such measurement is performed by metagenomic DNA sequencing. In another embodiment, the measurement is performed by RT-PCT counting. In a specific embodiment, bacterial housekeeping maker gene rpoB is used in the RT-PCT counting. In another embodiment, the measurement is performed by full length 16S RNA sequencing.

It has been observed in the present invention that above described health benefits are instigated by adding a selective feed additives to the feed of production animals. These additives are precision compounds which selectively modulate the composition and functions of the microbiome with the host animal. This selective modulation of the microbiome targets pathogenic E. coli bacteria and their coordinator B. thetaiotaomicron in the microbiome of the host animal.

In an embodiment, the feed additives are oligosaccharides. In the preferred embodiment, the oligosaccharides include but are not limited to glycan, yeast cell wall product and/or synthetic oligosaccharide preparation. In another preferred embodiment, the oligosaccharides are a synthetic oligosaccharide preparation, wherein said synthetic oligosaccharide preparation comprises at least n fractions of oligosaccharides each having a distinct degree of polymerization selected from 1 to n (DP1 to DPn fractions), wherein n is an integer greater than or equal to 2; and wherein each fraction comprises from at least about 0.5% to about 90% (e.g. from 1% to 90%; or e.g. from about 0.5% to about 15%) of anhydro-subunit containing oligosaccharides by relative abundance as determined by mass spectrometry. In order to produce the health benefits described in this application, a suitable amount of oligosaccharides is required depending on the type of animal and its stage of growth.

However, a minimal amount of oligosaccharides is required in order to obtain the health benefits. In one embodiment, the oligosaccharides are at least 200 mg/L of the feed. In another embodiment, the oligosaccharides are at least 400 mg/L of the feed. In one embodiment, the oligosaccharides are between 200 and 2000 mg/L of the feed. In one embodiment, the concentration of the oligosaccharides is at least 50 ppm (e.g. at least 50, 70, 100, 150, 200, 300, 400, 500 ppm) of the feed to be given to the group of production animals

In some embodiments, the oligosaccharide preparation comprises at least n fractions of oligosaccharides each having a distinct degree of polymerization selected from 1 to n (DP1 to DPn fractions), wherein n is an integer greater than or equal to 2; and each fraction comprises from at least about 0.5% to about 90% (e.g. from 1% to 90%; or e.g. from about 0.5% to about 15%) of anhydro-subunit containing oligosaccharides by relative abundance as determined by mass spectrometry.

In some embodiments, at least one fraction of the oligosaccharide preparation comprises less than 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, or 2% anhydro-subunit containing oligosaccharides by relative abundance; and/or wherein each fraction of the oligosaccharide preparation comprises greater than 0.2%, 0.5%, 1%, 2%, 3%, 4%, 5%, 6%, 7%, 8%, 9%, 10%, 11%, 12%, 13%, 14%, 15%, 16%, 17%, 18%, 19%, 20%, 30%, 40%, 50%, 60%, 70%, or 80% anhydro-subunit containing oligosaccharides by relative abundance.

In some embodiments, the oligosaccharide preparation has a weight average molecular weight from about 300 to 5000 g/mol (e.g. from about 2000 to 2800 g/mol, 2100 to 2700 g/mol, 2200 to 2600 g/mol, 2300 to 2500 g/mol, or 2320 to 2420 g/mol), 500 to 5000 g/mol, 700 to 5000 g/mol, 500 to 2000 g/mol, 700 to 2000 g/mol, 700 to 1500 g/mol, 300 to 1500 g/mol, 300 to 2000 g/mol, 400 to 1300 g/mol, 400 to 1200 g/mol, 400 to 1100 g/mol, 500 to 1300 g/mol, 500 to 1200 g/mol, 500 to 1100 g/mol, 600 to 1300 g/mol, 600 to 1200 g/mol, or 600 to 1100 g/mol; and/or wherein the oligosaccharide preparation has a number average molecular weight from about 1000 to 2000 g/mol, 1100 to 1900 g/mol, 1200 to 1800 g/mol, 1300 to 1700 g/mol, 1400 to 1600 g/mol, or 1450 to 1550 g/mol.

In some embodiments, the relative abundance of oligosaccharides in each of the n fractions of the oligosaccharide preparation decreases monotonically with its degree of polymerization.

In some embodiments, the relative abundance of oligosaccharides in at least 5, 10, 20, or 30 DP fractions of the oligosaccharide preparation decreases monotonically with its degree of polymerization.

In some embodiments, the oligosaccharide preparation comprises less than 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, or 2% anhydro-subunit containing oligosaccharides by relative abundance.

In some embodiments, each fraction of the oligosaccharide preparation comprises less than 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, or 2% anhydro-subunit containing oligosaccharides by relative abundance.

In some embodiments, at least one fraction of the oligosaccharide preparation comprises greater than 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 30%, 40%, 50%, 60%, 70%, or 80% anhydro-subunit containing oligosaccharides by relative abundance.

In some embodiments, the oligosaccharide preparation comprises greater than 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, or 25%, 30%, 40%, 50%, 60%, 70%, or 80% anhydro-subunit containing oligosaccharides by relative abundance.

In some embodiments, each fraction of the oligosaccharide preparation comprises greater than 20%, 21%, 22%, 23%, 24%, or 25% anhydro-subunit containing oligosaccharides by relative abundance.

In some embodiments, more than 99%, 95%, 90%, 85%, 80%, 75%, 70%, 65%, 60%, 55%, 50%, 45%, 40%, 35%, or 30% of the anhydro-subunit containing oligosaccharides of the oligosaccharide preparation have only one anhydro-subunit.

In some embodiments, the oligosaccharide preparation has a DP1 fraction content from 1 to 40% by relative abundance.

In some embodiments, the oligosaccharide preparation has a DP2 fraction content from 1 to 35% by relative abundance.

In some embodiments, the oligosaccharide preparation has a DP3 fraction content from 1 to 30% by relative abundance.

In some embodiments, the oligosaccharide preparation has a DP4 fraction content from 0.1 to 20% by relative abundance.

In some embodiments, the oligosaccharide preparation has a DP5 fraction content from 0.1 to 15% by relative abundance.

In some embodiments, the ratio of DP2 fraction to DP1 fraction of the oligosaccharide preparation is 0.02-0.40 by relative abundance.

In some embodiments, the ratio of DP3 fraction to DP2 fraction of the oligosaccharide preparation is 0.01-0.30 by relative abundance.

In some embodiments, the aggregate content of DP1 and DP2 fractions in the oligosaccharide preparation is less than 50, 30, or 10% by relative abundance.

In some embodiments, the oligosaccharide preparation comprises at least 103, 104, 105, 106 or 109 different oligosaccharide species.

In some embodiments, two or more independent oligosaccharides of the oligosaccharide preparation comprise different anhydro-subunits.

In some embodiments, the oligosaccharide preparation comprises one or more anhydro-subunits that are products of reversible thermal dehydration of monosaccharides.

In some embodiments, the oligosaccharide preparation comprises one or more anhydro-glucose, anhydro-galactose, anhydro-mannose, anhydro-allose, anhydro-altrose, anhydro-gulose, anhydro-indose, anhydro-talose, anhydro-fructose, anhydro-ribose, anhydro-arabinose, anhydro-rhamnose, anhydro-lyxose, or anhydro-xylose subunits.

In some embodiments, the oligosaccharide preparation comprises one or more anhydro-glucose, anhydro-galactose, anhydro-mannose, or anhydro-fructose subunits.

In some embodiments, the oligosaccharide preparation comprises one or more 1,6-anhydro-p-D-glucofuranose or 1,6-anhydro-p-D-glucopyranose subunits. In some embodiments, the oligosaccharide preparation comprises both 1,6-anhydro-p-D-glucofuranose and 1,6-anhydro-p-D-glucopyranose anhydro-subunits.

In some embodiments, a ratio of 1,6-anhydro-p-D-glucofuranose to 1,6-anhydro-P-D-glucopyranose is from about 10:1 to 1:10, 9:1 to 1:10, 8:1 to 1:10, 7:1 to 1:10, 6:1 to 1:10, 5:1 to 1:10, 4:1 to 1:10, 3:1 to 1:10, 2:1 to 1:10, 10:1 to 1:9, 10:1 to 1:8, 10:1 to 1:7, 10:1 to 1:6, 10:1 to 1:5, 10:1 to 1:4, 10:1 to 1:3, 10:1 to 1:2, or 1:1 to 3:1 in the oligosaccharide preparation.

In some embodiments, the ratio of 1,6-anhydro-p-D-glucofuranose to 1,6-anhydro-p-D-glucopyranose is about 10:1, 9:1, 8:1, 7:1, 6:1, 5:1, 4:1, 3:1, 2:1, 1:1, 1:2, 1:3, 1:4, 1:5, 1:6, 1:7, 1:8, 1:8, 1:9, or 1:10 within the oligosaccharide preparation.

In some embodiments, the ratio of 1,6-anhydro-p-D-glucofuranose to 1,6-anhydro-p-D-glucopyranose is about 2:1 in the oligosaccharide preparation.

In some embodiments, the ratio of 1,6-anhydro-p-D-glucofuranose to 1,6-anhydro-p-D-glucopyranose is about from 10:1 to 1:10, 9:1 to 1:10, 8:1 to 1:10, 7:1 to 1:10, 6:1 to 1:10, 5:1 to 1:10, 4:1 to 1:10, 3:1 to 1:10, 2:1 to 1:10, 10:1 to 1:9, 10:1 to 1:8, 10:1 to 1:7, 10:1 to 1:6, 10:1 to 1:5, 10:1 to 1:4, 10:1 to 1:3, 10:1 to 1:2, or 1:1 to 3:1 in each fraction of the oligosaccharide preparation.

In some embodiments, the ratio of 1,6-anhydro-p-D-glucofuranose to 1,6-anhydro-p-D-glucopyranose is about 10:1, 9:1, 8:1, 7:1, 6:1, 5:1, 4:1, 3:1, 2:1, 1:1, 1:2, 1:3, 1:4, 1:5, 1:6, 1:7, 1:8, 1:8, 1:9, or 1:10 in each fraction of the oligosaccharide preparation.

In some embodiments, the ratio of 1,6-anhydro-p-D-glucofuranose to 1,6-anhydro-p-D-glucopyranose is about 2:1 in each fraction of the oligosaccharide preparation.

In some embodiments, at least 1%, 5%, 10%, 20%, 30%, 40%, 50%, 60%, 70%, 80%, or 90% of anhydro-subunits in the oligosaccharide preparation are selected from a group consisting of 1,6-anhydro-p-D-glucofuranose and 1,6-anhydro-p-D-glucopyranose.

In some embodiments, the weight average molecular weight of the oligosaccharide preparation is about from 300 to 5000 g/mol, 500 to 5000 g/mol, 700 to 5000 g/mol, 500 to 2000 g/mol, 700 to 2000 g/mol, 700 to 1500 g/mol, 300 to 1500 g/mol, 300 to 2000 g/mol, 400 to 1300 g/mol, 400 to 1200 g/mol, 400 to 1100 g/mol, 500 to 1300 g/mol, 500 to 1200 g/mol, 500 to 1100 g/mol, 600 to 1300 g/mol, 600 to 1200 g/mol, or 600 to 1100 g/mol.

In some embodiments, the number average molecular weight of the oligosaccharide preparation is about from 300 to 5000 g/mol, 500 to 5000 g/mol, 700 to 5000 g/mol, 500 to 2000 g/mol, 700 to 2000 g/mol, 700 to 1500 g/mol, 300 to 1500 g/mol, 300 to 2000 g/mol, 400 to 1000 g/mol, 400 to 900 g/mol, 400 to 800 g/mol, 500 to 900 g/mol, or 500 to 800 g/mol.

In some embodiments, the distribution of the degree of polymerization is determined and/or detected by MALDI-MS, GC-MS, LC-MS, SEC, HPLC and/or combination(s) thereof (e.g. MALDI-MS and SEC).

In some embodiments, the degree of polymerization of the oligosaccharide preparation may be determined based on its molecular weight and molecular weight distribution.

The oligosaccharide preparation referred to herein may be characterized by any one, two or more or even all of the individual features of the oligosaccharide preparation as described above.

In other words, the oligosaccharide preparation may be characterized by any combination of the individual features as described in the items above. For instance, in a particular embodiment of a method according to the invention, the oligosaccharide preparation may be characterized by a combination of the combined oligosaccharide preparation features, which are that the relative abundance of oligosaccharides in at least 5, 10, 20, or 30 DP fractions of the oligosaccharide preparation decreases monotonically with its degree of polymerization; that the oligosaccharide preparation has a DP2 fraction content from 1 to 35% by relative abundance; that the aggregate content of DP1 and DP2 fractions in the oligosaccharide preparation is less than 50, 30, or 10% by relative abundance; and that the ratio of 1,6-anhydro-p-D-glucofuranose to 1,6-anhydro-p-D-glucopyranose is about 2:1 in the oligosaccharide preparation.

In some embodiments, the oligosaccharide preparation is comprised in the nutritional composition at a concentration of at least 50 g per ton of feed (e.g. at least 70 g, 100 g, 200 g, 300 g, 400 g, 500 g, 600 g, 700 g, 800 g, 900 g per ton of feed); and/or wherein the oligosaccharide preparationis comprised in the nutritional composition at an inclusion rate of at least 50 ppm (e.g. at least 50, 70, 100, 150, 200, 300, 400, 500 ppm); and/or wherein the oligosaccharide preparation is comprised in the nutritional composition at a concentration of at least 50 ppm (e.g. at least 50, 70, 100, 150, 200, 300, 400, 500 ppm).

In some embodiments, the oligosaccharide preparation is administered for at least one day, preferably for at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, or 245 days, most preferably, the nutritional composition is administered continuously, i.e. uninterruptedly.

The oligosaccharide preparation may be provided in the form of a powderous formulation comprising at least 20% (w/w) of the oligosaccharide preparation as referred to herein; at least 25% (wt/wt) of a silica-based adsorbate (e.g. diatomaceous earth, amorphous precipitated silica) having an average particle size D of less than or equal to 3000 μm (e.g. 100-500, 200-500, 200-300 μm); and optionally 0-25% (wt/wt) of water and/or an auxiliary substance; wherein the % are based on the total weight of the powderous formulation. For instance, such a powderous formulation may comprise 30-70% (wt/wt) of the oligosaccharide preparation as referred to herein; 30-70% (wt/wt) of a silica based adsorbate (e.g. having an average particle size of at least 50 μm); and 0-21% (wt/wt) of water; wherein the % are based on the total weight of the powderous formulation. In some embodiments the oligosaccharide preparation is formulated as described in any one of Examples 22-26 and 33 of WO 2020/097458, which is incorporated by reference herein.

In another embodiment, the feed additives are essential oils. In order to produce the health benefits described in this application, a suitable amount of essential oils is required depending on the type of animal and its stage of growth. However, a minimal amount of essential oils is required in order to obtain the health benefits. In some embodiment, the essential oils is at 200 ppm, at least 250 ppm, at least 300 ppm, at least 350 ppm, at least 400 ppm. At least 450 ppm, or at least 500 ppm of the feed, In some embodiments, the concentration of the essential oil in the feed is between 100-1000 ppm, between 100-800 ppm, between 100-600 ppm, between 200-500 ppm, between 200-400 ppm.

In some embodiments, the invention relates to a use of oligosaccharides (e.g. glycans, yeast cell walls, and/or (synthetic) oligosaccharide preparations), and/or essential oils for a) reducing the population of exogenous locus for enterocyte effacement (LEE) genes and exogenous non-LEE pathogenic genes of Enterohemorrhagic E. coli(EHEC), Enteropathogenic E. coli(EPEC), and Avian Pathogenic E. coli (APEC) in the gastrointestinal tract (GIT) of an animal, wherein the population of exogenous LEE genes and non-LEE pathogenic genes is reduced by at least 10% lower than that of a control animal which is fed with the same diet except for said feed additives; b) reducing the population of Bacteroides thetaiotaomicron in the gastrointestinal tract (GIT) of an animal, wherein the population of Bacteroides thetaiotaomicron is reduced by at least 10% lower than that of a control animal which is fed with the same diet except for said feed additives; c) reducing the population of E. coli in the gastrointestinal tract (GIT) of an animal, wherein the systemic inflammation and/or local inflammation of the animal is reduced by at least 10% lower than that of a control animal which are fed with the same diet except for said feed additives; and/or for d) reducing systemic inflammation and/or local inflammation of an animal caused by E. coli infection, wherein the systemic inflammation and/or local inflammation of the animal is reduced by at least 10% lower than that of a control animal which are fed with the same diet except for said feed additives. In some embodiments, said use relates to the use of an oligosaccharide preparation for a) reducing the population of exogenous locus for enterocyte effacement (LEE) genes and exogenous non-LEE pathogenic genes of Enterohemorrhagic E. coli (EHEC), Enteropathogenic E. coli (EPEC), and Avian Pathogenic E. coli (APEC) in the gastrointestinal tract (GIT) of an animal, wherein the population of exogenous LEE genes and non-LEE pathogenic genes is reduced by at least 10% lower than that of a control animal which is fed with the same diet except for said feed additives; b) reducing the population of Bacteroides thetaiotaomicron in the gastrointestinal tract (GIT) of an animal, wherein the population of Bacteroides thetaiotaomicron is reduced by at least 10% lower than that of a control animal which is fed with the same diet except for said feed additives; c) reducing the population of E. coli in the gastrointestinal tract (GIT) of an animal, wherein the systemic inflammation and/or local inflammation of the animal is reduced by at least 10% lower than that of a control animal which are fed with the same diet except for said feed additives; and/or for d) reducing systemic inflammation and/or local inflammation of an animal caused by E. coli infection, wherein the systemic inflammation and/or local inflammation of the animal is reduced by at least 10% lower than that of a control animal which are fed with the same diet except for said feed additives; wherein said oligosaccharide preparation comprises at least n fractions of oligosaccharides each having a distinct degree of polymerization selected from 1 to n (DP1 to DPn fractions), wherein n is an integer greater than or equal to 2; and wherein each fraction comprises from at least about 0.5% to about 90% (e.g. from 1% to 90%; or e.g. from about 0.5% to about 15%) of anhydro-subunit containing oligosaccharides by relative abundance as determined by mass spectrometry.

### Type of Animal

The method of the present invention is applicable to production animals in general. In one embodiment, the method of the present invention is applicable to poultry.

The above mentioned feed additives may be provided to any suitable animal. In some embodiments, the animal is monogastric. It is generally understood that a monogastric animal has a single-chambered stomach. In other embodiments, the animal is a ruminant. It is generally understood that a ruminant has a multi-chambered stomach. In some embodiments, the animal is a ruminant in the pre-ruminant phase. Examples of such ruminants in the pre-ruminant phase include nursery calves.

In some embodiments, the animal is a poultry (e.g. chicken, turkey), seafood (e.g. shrimp), sheep, cow, cattle, buffalo, bison, pig (e.g. nursery pig, grower/finisher pig), cat, dog, rabbit, goat, guinea pig, donkey, camel, horse, pigeon, ferret, gerbil, hamster, mouse, rat, bird, or human.

In some embodiments, the animal is livestock. In some embodiments, the animal is a companion animal. In some embodiments, the animal is poultry. Examples of poultry include chicken, duck, turkey, goose, quail, or Cornish game hen. In one variation, the animal is a chicken. In some embodiments, the poultry is a layer hen, a broiler chicken, or a turkey.

In other embodiments, the animal is a mammal, including, for example, a cow, a pig, a goat, a sheep, a deer, a bison, a rabbit, an alpaca, a llama, a mule, a horse, a reindeer, a water buffalo, a yak, a guinea pig, a rat, a mouse, an alpaca, a dog, or a cat. In one variation, the animal is a cow. In another variation, the animal is a pig. In another variation, the animal is a sow.

### Administration of Feed Additives

In some embodiments, administration comprises providing the feed additives described herein to an animal such that the animal may ingest the feed additives at will. In such embodiments, the animal ingests some portion of the feed additives.

The feed additives described herein may be provided to the animal on any appropriate schedule. In some embodiments, the animal is the feed additives described herein on a daily basis, on a weekly basis, on a monthly basis, on an every other day basis, for at least three days out of every week, or for at least seven days out of every month.

In some embodiments, the feed additives described herein is administered to the animal multiple times in a day. For examples, in some embodiments, the feed additives described herein is administered to the animal at least 1, 2, 3, 4, 5, 6, 7, 8, 9, or 10 times a day. In some embodiments, the nutritional composition, the feed additives described herein is administered to the animal at most 1, 2, 3, 4, 5, 6, 7, 8, 9, or 10 times a day.

In some embodiments, the feed additives described herein is administered to the animal multiple times in a day. For examples, in some embodiments, the feed additives described herein is administered to the animal at least 1, 2, 3, 4, 5, 6, 7, 8, 9, or 10 times a week. In some embodiments, the nutritional composition, the feed additives described herein is administered to the animal at most 1, 2, 3, 4, 5, 6, 7, 8, 9, or 10 times a week. In some embodiments, the feed additives described herein is administered to the animal every day, every other day, every 3 days, every 4 days, every week, every other week, or every month.

In some embodiments, the animal is the feed additives described herein during certain diet phases. For example, some animals are provided a starter diet between 0 to 14 days of age. In other embodiments, an animal is provided a grower diet between 15 to 28 days of age, between 15 to 35 days of age, or between 15 to 39 days of age. In still other embodiments, an animal is provided a finisher diet between 29 to 35 days of age, between 36 to 42 days of age, or between 40 to 46 days of age.

In certain embodiments, the feed additives described herein is provided to the animal during the starter diet phase, the grower diet phase, or the finisher diet phase, or any combinations thereof.

In certain embodiments, the animal is poultry, and the poultry is provided a starter diet between 0 to 15 days of age, a grower diet between 16 to 28 days of age, and a finisher diet between 29 to 35 days of age. In other embodiments, the animal is poultry, and the poultry is provided a starter diet between 0 to 14 days of age, a grower diet between 15 to 35 days of age, and a finisher diet between 36 to 42 days of age. In still other embodiments, the animal is poultry, and the poultry is provided a starter diet between 0 to 14 days of age, a grower diet between 15 to 39 days of age, and a finisher diet between 20 to 46 days of age.

In some embodiments, the feed additives described herein is provided to the poultry during the starter diet phase, the grower diet phase, or the finisher diet phase, or any combinations thereof.

The feed additives described herein may be fed to individual animals or an animal population. For example, in one variation where the animal is poultry, the feed additives described herein may be fed to an individual poultry or a poultry population.

The feed additives described herein may be provided to an animal in any appropriate form, including, for example, in solid form, in liquid form, or a combination thereof. In certain embodiments, the feed additives described herein is a liquid, such as a syrup or a solution. In other embodiments, the feed additives described herein is a solid, such as pellets or powder. In yet other embodiments, the feed additives described herein may be fed to the animal in both liquid and solid components, such as in a mash.

The present invention can be further characterized by the following embodiments:

Item 1: A method for reducing the population of exogenous locus for enterocyte effacement (LEE) genes and exogenous non-LEE pathogenic genes of Enterohemorrhagic E. coli (EHEC), Enteropathogenic E. coli (EPEC), and Avian Pathogenic E. coli (APEC) in the gastrointestinal tract (GIT) of an animal, comprising feeding said animal with one of more of the following feed additives: oligosaccharides, and essential oils, wherein the population of exogenous LEE genes and non-LEE pathogenic genes is reduced by at least 10% lower than that of a control animal which is fed with the same diet except for said feed additives.

Item 2: The method of Item 1, wherein said population of exogenous LEE genes and non-LEE pathogenic genes is measured as % ratio of the combined copy numbers of LEE genes and non-LEE genes detected within the microbiome of said animal vs. the total copy number of genes detected within said microbiome.

Item 3: The method of Item 2, wherein said microbiome is collected from either a fecal sample of the animal or a sample collected within the GIT of the animal.

Item 4: The method of Item 3, wherein said gene copy number measurement is performed by RT-PCR counting, full length 16S RNA sequencing, or Metagenomic DNA sequencing.

Item 5: The method of any one of Items 1-4, wherein said animal is a production animal.

Item 6: The method of any one of Items 1-5, wherein said LEE genes comprise: Tir, Map, EspB, EspF, EspG, EspH, and EspZ.

Item 7: The method of any one of Items 1-5, wherein said non-LEE pathogenic genes comprise: EspG2, EspJ, EspM1/2, EspT, EspW, Cif, NleA, NleB, NleC, NIeD, NleE, NIeF, and NleH.

Item 8: The method of any one of Items 1-7, wherein said oligosaccharides are glycans, yeast cell walls, and/or synthetic oligosaccharide preparation, wherein said synthetic oligosaccharide preparation comprises at least n fractions of oligosaccharides each having a distinct degree of polymerization selected from 1 to n (DP1 to DPn fractions), wherein n is an integer greater than or equal to 2; and wherein each fraction comprises from at least about 0.5% to about 90% (e.g. from 1% to 90%; or e.g. from about 0.5% to about 15%) of anhydro-subunit containing oligosaccharides by relative abundance as determined by mass spectrometry.

Item 9: The method of item 8, wherein the concentration of said oligosaccharides is between 200 and 2000 mg/L of the feed or at least 50 ppm (e.g. at least 50, 70, 100, 150, 200, 300, 400, 500 ppm) of the feed to be given to the group of production animals.

Item 10: The method of any one of Items 1-7, wherein the concentration of said essential oils is between 100-1000 ppm of the feed to be given to the group of production animals.

Item 11: The method of Items 1-10, wherein said production animals are: broiler chickens, turkeys, ducks, layers, piglets, grower pigs, finisher pigs, and sows.

Item 12: A method for reducing the population of Bacteroides thetaiotaomicron in the gastrointestinal tract (GIT) of an animal, comprising feeding said animal with one of more of the following feed additives: oligosaccharides, and essential oils, wherein the population of Bacteroides thetaiotaomicron is reduced by at least 10% lower than that of a control animal which is fed with the same diet except for said feed additives.

Item 13: The method of Item 12, wherein said population of Bacteroides thetaiotaomicron is measured as % ratio of the population of Bacteroides thetaiotaomicron detected within the microbiome of said animal against the total population of microbes within said microbiome.

Item 14: The method of Item 13, wherein said microbiome is collected from the fecal sample of the animal or a sample collected within the GIT of the animal.

Item 15: The method of Item 14, wherein population measurement is performed by RT-PCT counting, full length 16S RNA sequencing, or Metagenomic DNA sequencing.

Item 16: The method of any one of Items 12-15, wherein said animal is a production animal.

Item 17: The method of any one of Items 12-16, wherein said oligosaccharides are glycans, yeast cell walls, and/or synthetic oligosaccharide preparation, wherein said synthetic oligosaccharide preparation comprises at least n fractions of oligosaccharides each having a distinct degree of polymerization selected from 1 to n (DP1 to DPn fractions), wherein n is an integer greater than or equal to 2; and wherein each fraction comprises from at least about 0.5% to about 90% (e.g. from 1% to 90%; or e.g. from about 0.5% to about 15%) of anhydro-subunit containing oligosaccharides by relative abundance as determined by mass spectrometry.

Item 18: The method of item 17, wherein the concentration of said oligosaccharides is between 200 and 2000 mg/L of the feed or at least 50 ppm (e.g. at least 50, 70, 100, 150, 200, 300, 400, 500 ppm) of the feed to be given to the group of production animals.

Item 19: A method of reducing the population of E. coli in the gastrointestinal tract (GIT) of an animal, comprising feeding said animal with one of more of the following feed additives: oligosaccharides, and essential oils, wherein the systemic inflammation and/or local inflammation of the animal is reduced by at least 10% lower than that of a control animal which are fed with the same diet except for said feed additives.

Item 20: The method of Item 17, wherein said E. coli is pathogenic E. coli.

Item 21: The method of Item 18, wherein the pathogenic E. coli comprises EPEC, EHEC, and APEC.

Item 22: The method of Item 19, wherein said population of E. coli in the GIT of the animal is measured as % of copy number of E. coli marker genes within the microbiome of said animal against the total copy number of bacterial marker genes detected within said microbiome.

Item 23: The method of Item 20, wherein said microbiome is collected from the fecal sample of the animal or a sample collected within the GIT of the animal.

Item 24: The method of Item 21, wherein said measurement is performed by RT-PCT counting, full length 16S RNA sequencing, or Metagenomic DNA sequencing.

Item 25: The method of any one of Items 17-22, wherein said animal is a production animal.

Item 26: The method of any one of Items 19-25, wherein said oligosaccharides are glycans, yeast cell walls, and/or synthetic oligosaccharide preparation, wherein said synthetic oligosaccharide preparation comprises at least n fractions of oligosaccharides each having a distinct degree of polymerization selected from 1 to n (DP1 to DPn fractions), wherein n is an integer greater than or equal to 2; and wherein each fraction comprises from at least about 0.5% to about 90% (e.g. from 1% to 90%; or e.g. from about 0.5% to about 15%) of anhydro-subunit containing oligosaccharides by relative abundance as determined by mass spectrometry.

Item 27: The method of item 26, wherein the concentration of said oligosaccharides is between 200 and 2000 mg/L of the feed or at least 50 ppm (e.g. at least 50, 70, 100, 150, 200, 300, 400, 500 ppm) of the feed to be given to the group of production animals.

Item 28: A method for reducing systemic inflammation and/or local inflammation of an animal caused by E. coli infection, comprising feeding said animal with one of more of the following feed additives: oligosaccharides, and essential oils, wherein the systemic inflammation and/or local inflammation of the animal is reduced by at least 10% lower than that of a control animal which are fed with the same diet except for said feed additives.

Item 29: The method of Item 24, wherein said reduction of inflammation is measured as % ratio of the copy number of LEE and non-LEE genes detected within the microbiome of said animal against the total copy number of genes detected within said microbiome.

Item 30: The method of Item 25, wherein said microbiome is collected from the fecal sample of the animal or a sample collected within the GIT of the animal.

Item 31: The method of Item 26, wherein said measurement is performed by RT-PCT counting, full length 16S RNA sequencing, or Metagenomic DNA sequencing.

Item 32: The method of any one of Items 24-27, wherein said animal is a production animal.

Item 33: The method of any one of Items 28-32, wherein said oligosaccharides are glycans, yeast cell walls, and/or synthetic oligosaccharide preparation, wherein said synthetic oligosaccharide preparation comprises at least n fractions of oligosaccharides each having a distinct degree of polymerization selected from 1 to n (DP1 to DPn fractions), wherein n is an integer greater than or equal to 2; and wherein each fraction comprises from at least about 0.5% to about 90% (e.g. from 1% to 90%; or e.g. from about 0.5% to about 15%) of anhydro-subunit containing oligosaccharides by relative abundance as determined by mass spectrometry.

Item 34: The method of item 33, wherein the concentration of said oligosaccharides is between 200 and 2000 mg/L of the feed or at least 50 ppm (e.g. at least 50, 70, 100, 150, 200, 300, 400, 500 ppm) of the feed to be given to the group of production animals.

Item 35: Use of oligosaccharides (e.g. glycans, yeast cell walls, and/or (synthetic) oligosaccharide preparations), and/or essential oils for
a) reducing the population of exogenous locus for enterocyte effacement (LEE) genes and exogenous non-LEE pathogenic genes of Enterohemorrhagic E. coli (EHEC), Enteropathogenic E. coli (EPEC), and Avian Pathogenic E. coli (APEC) in the gastrointestinal tract (GIT) of an animal, wherein the population of exogenous LEE genes and non-LEE pathogenic genes is reduced by at least 10% lower than that of a control animal which is fed with the same diet except for said feed additives;
b) reducing the population of Bacteroides thetaiotaomicron in the gastrointestinal tract (GIT) of an animal, wherein the population of Bacteroides thetaiotaomicron is reduced by at least 10% lower than that of a control animal which is fed with the same diet except for said feed additives; c) reducing the population of E. coli in the gastrointestinal tract (GIT) of an animal, wherein the systemic inflammation and/or local inflammation of the animal is reduced by at least 10% lower than that of a control animal which are fed with the same diet except for said feed additives; and/or d) reducing systemic inflammation and/or local inflammation of an animal caused by E. coli infection, wherein the systemic inflammation and/or local inflammation of the animal is reduced by at least 10% lower than that of a control animal which are fed with the same diet except for said feed additives.

Item 36: Use of an oligosaccharide preparation for
a) reducing the population of exogenous locus for enterocyte effacement (LEE) genes and exogenous non-LEE pathogenic genes of Enterohemorrhagic E. coli (EHEC), Enteropathogenic E. coli (EPEC), and Avian Pathogenic E. coli (APEC) in the gastrointestinal tract (GIT) of an animal, wherein the population of exogenous LEE genes and non-LEE pathogenic genes is reduced by at least 10% lower than that of a control animal which is fed with the same diet except for said feed additives;
b) reducing the population of Bacteroides thetaiotaomicron in the gastrointestinal tract (GIT) of an animal, wherein the population of Bacteroides thetaiotaomicron is reduced by at least 10% lower than that of a control animal which is fed with the same diet except for said feed additives; c) reducing the population of E. coli in the gastrointestinal tract (GIT) of an animal, wherein the systemic inflammation and/or local inflammation of the animal is reduced by at least 10% lower than that of a control animal which are fed with the same diet except for said feed additives; and/or d) reducing systemic inflammation and/or local inflammation of an animal caused by E. coli infection, wherein the systemic inflammation and/or local inflammation of the animal is reduced by at least 10% lower than that of a control animal which are fed with the same diet except for said feed additives, wherein the systemic inflammation and/or local inflammation of the animal is reduced by at least 10% lower than that of a control animal which are fed with the same diet except for said feed additives; wherein said oligosaccharide preparation comprises at least n fractions of oligosaccharides each having a distinct degree of polymerization selected from 1 to n (DP1 to DPn fractions), wherein n is an integer greater than or equal to 2; and wherein each fraction comprises from at least about 0.5% to about 90% (e.g. from 1% to 90%; or e.g. from about 0.5% to about 15%) of anhydro-subunit containing oligosaccharides by relative abundance as determined by mass spectrometry.

### EXAMPLES

### Example 1

Example 1 of the study describes the protocols and methods used for generating and analyzing the data in this invention.

### Sample Collection

Cecal digesta samples were collected at various days depending on the their species and growing schedule from both Negative Control and treatment groups (1 bird/pen and 21 replicates/treatment). Cecal samples were kept frozen at −80° C. before DNA extraction for metagenomics or solvent extraction for metabolomics analysis.

DNA extraction and Sequencing

Quantitative measurement of gene copies can be made through any shotgun sequencing measurement method. In this application, metagenomic DNA was extracted using MoBio Powersoil following manufacturer instructions (Qiagen, Germany). DNA was sequenced at Diversigen (TX, USA), on an Illumina HiSeq 3000 apparatus with a target depth of 5 GB per sample.

### Taxonomy Read Processing

In order to choose appropriate filtering and trimming parameters, the raw fastq files from shallow 122 shotgun sequencing were inspected using FastQC v0.11.5. Based on the quality reports, Cutadapt was used to trim the first 10 bases of each read, shorten each read to a maximum of 130 bp, and discard any read less than 120 bp long. This removed any remaining adapter fragments and eliminated regions near the end of the read where the quality dropped, confirmed by another quality report from FastQC.

### Taxonomy Analysis

Sequences read from the instrument can then be aligned using any alignment algorithm against a reference database of genes containing at a minimal, LEE and non-LEE genes. In the present application, MetaPhlan 2.0, analysis type “rel_ab_w_read_stats” was used to construct a profile of taxonomic relative abundance for each sample from the processed reads using forward reads only.

### Functional Mapping

The processed reads were mapped against an internal gene catalog specifically tailored to the chicken gut microbiome with bwa v0.7.5 using the BWA-MEM algorithm. Python scripts were used to extract a table of gene counts for each sample from the BAM files, which was used as the input for downstream analysis. Only reads that mapped in a proper pair were considered a successful hit to a gene. An internal gene catalog has been annotated using the publicly available KEGG Orthology (KO) database, and much of the metagenomics analysis discussed here is based on functional information from KEGG (Kyoto Encyclopedia of Genes and Genomes).

### Example 2

A feeding trial was performed to study the effects of oligosaccharide preparations on birds in husbandry. The test period began on Trial Day 0 (day of hatch of chicks), when chicks began being fed a commercial-type feed in pelleted form (further crumbled for Starter feeds), and ended on Trial Day 42. Each experimental unit contained 40 male broilers (Hubbard-Cobb) randomly assigned into 21 replicates per group for a total number of 840 animals per treatment on study. Broiler chicks were randomly assigned to treatments of Trial Day 0 (or on day of hatch) and were not replaced during the course of the trial. The chicks were observed daily for signs of unusual grow-out patterns or health problems. Body weights, feed consumption and feed conversion were measured on Trial Days 0, 10, 24, and 42. Cecal content samples, ileal tissue samples, and blood plasma samples were collected from 1 bird per pen at 24 and 42 days of age. For the vaccination program, all birds received Marek's vaccine, as well as being sprayed with vaccine against coccidiosis (COCCIVAC®-B52 by Merck Animal Health USA, which is a live oocysts vaccine isolated from chickens, prepared from anticoccidial-sensitive strains of E. acervulina, E. maxima, E. maxima MFP, E. mivati, and E. tenella according to the product bulletin) and for Newcastle bronchitis. No feed grade antibiotics were administered during the course of the study. All birds were grown on new litter. Feed and water were provided ad libitum throughout the conduct of the study.

The commercial-simulated test model employed in this study used broiler chicks (Gallus gallus domesticus) reared under a normal poultry industry Starter diet (0-10 days of age), Grower diet (11-24 days of age) and Finisher diet (25-42 days of age) at a floor space requirement of a minimum of 0.85 ft² per bird, reared in floor pens with new litter. Ration formulations were conducted via computer-generated linear regression program that simulates formulations conducted during practical poultry production techniques. Treatments were tested in male broilers. Broilers were continuously fed their experimental diets from time of placement on Trial Day 0 (day of hatch) to 42 days of age. All diets contained 1000 FYT/kg of phytase (RONOZYME® HiPhos).

| | | Test Material |
|---|---|---|
| | | Inclusion |
| Treatment | Description | (ppm) |
| Control | high/poor quality protein | 0 |
| Test | Control + oligosaccharide | 500 |
| | preparation | |

Broiler chicks were weighed and randomly placed into each pen on day of hatch (Trial Day 0) and fed their respective diets. Each pen had sufficient floor density, feeder and waterer space for each grow-out area for chickens up to 42 days of age. Following 42 days of grow-out, broilers were weighed, feed consumption determined, and feed conversion ratio (feed consumed/body weight) calculated and adjusted for mortality.

Oligosaccharide preparations comprise at least n fractions of oligosaccharides each having a distinct degree of polymerization selected from 1 to n (DP1 to DPn fractions), wherein n is an integer greater than 3; wherein each of a DP1 and DP2 fraction independently comprises from about 0.5% to about 15% of anhydro-subunit containing oligosaccharides by relative abundance as determined by mass spectrometry. Oligosaccharide preparations were produced as described herein and as disclosed in WO 2020/097458, and WO 2016/007778, which are herein incorporated by reference, in particular in the Examples described therein, in particular in any one of Examples 1-7, 16-18 of WO 2020/097458 A1, in the methods described in paragraph [317], and/or in any one of Examples 73-77, 80-89, 97-99, 101-110 of WO 2016/007778 A1.

Test material description: Test material was provided in either liquid or powder form, and mixed into the treatment feeds. The treatment feeds were then pelleted (and further crumbled for the Starter feeds) and placed into the pens according to the pen design for this study. Treatments were fed continuously from Trial Days 0-42. Test material treatments (comprising oligosaccharide preparation according to the invention) were compared to a Control treatment (not comprising oligosaccharide preparation according to the invention).

Experimental design: A total of 8,000 male broiler chicks (a sufficient number to ensure availability of at least 7,560 healthy male chicks for the conduct of the study) were obtained from a commercial hatchery on Trial Day 0 (same as hatch date). These were immediately transported to the feeding trial facility under temperature-controlled conditions to assure bird comfort. After arrival at the facility, broilers were immediately randomized. There were 40 healthy/viable male broilers per pen with 21 pens per test group for a total of 840 broilers per treatment group. Broilers were fed their respective treatment feed ad libitum from day of hatch (Trial Day 0) to 42 days of age.

Detailed broiler chick description: Animal care practices conformed to the Guide for the Care and Use of Agricultural Animals in Agricultural Research and Teaching (FASS, 2010, 3rd Edition). Commercial broilers (Hubbard-Cobb) were obtained at hatch (Trial Day 0) from a commercial hatchery. Broilers were evaluated upon receipt for signs of disease or other complications that may have affected the outcome of the study. Following examination, broilers were weighed. Broilers were allocated to each pen and to treatment groups using a randomized block design. Weight distribution across the treatment groups were assessed prior to feeding by comparing the individual test group standard deviations of the mean against that of the control group. Differences between control and test groups were within one standard deviation, and as such, weight distribution across treatment groups were considered acceptable for this study.

Broiler chicks (on day of hatch, called Day 0) were collected in the early morning and were randomly assigned to each experimental pen within 12 hours of hatch. Weak birds were removed and humanely sacrificed. Birds were not replaced during the study.

Housing and daily observations: Each experimental test unit of broiler mixed-sex chicken pens were housed in separated pens, located in a room containing forced air heaters with a cross-house ventilation system. Broilers were placed in a 5 ft×10 ft pen floor area with a minimum of 0.85 ft² per bird (without feeder and waterer space) provided. At least two nipple drinkers per pen (via well water) were provided.

Feeders were employed for the grow-out period and checked daily to ensure that all birds had access to feed at all times.

The light program employed made use of incandescent lighting for approximately 23 hours of continuous light and 1 hour of darkness per day for Days 0-7, and for approximately 20 hours of continuous light and 4 hours of darkness per day for the remainder of the study.

Birds were observed daily for overall health, behavior and/or evidence of toxicity, and environmental conditions. Temperature in the test facility was checked daily. Drinking water and feed were confirmed to be provided ad libitum.

No type of medication (other than test material) was administered during the entire feeding period. Mortalities were collected daily and body weights recorded on all broilers found dead or moribund.

Data and observations: Live performance body weights and feed intakes were collected on Days 0, 10, 24, and 42 during the growing period. Weight gain, feed intake, feed:gain ratio (feed efficiency) were calculated for 0-42 days of age and other age periods between hatch and market weights. Differences between broilers fed control and test groups were statistically evaluated at P<0.05 in a typical ANOVA analysis of variance test model, employing Treatment×Replicate RCB (Randomized Complete Block). Control group was considered to be the following: Treatment 1, with no added test materials.

At the end of the study, all carcasses of necropsied broilers and all birds remaining at the end of the study, after being humanely euthanized, were disposed of according to local regulations via on-farm composting techniques.

Diet Preparation: A basal ration for each phase was formulated to meet or exceed minimum nutrient requirements of a typical commercial broiler diet using formulations employed by qualified nutritionist with training in poultry feed formulations, and formulated rations met or exceeded NRC Nutrient Requirements for Poultry as a guideline (9th edition, 1994). Feed formulations were furnished by a veterinarian, conducted by a regression analysis program commonly used for Least-Cost Feed Formulation in the poultry industry. Test materials were then mixed into the basal ration.

Dietary protein, lysine, methionine, methionine+cystine, arginine, threonine, tryptophan, total phosphorus, available phosphorus, total calcium, dietary sodium, and dietary choline were met by adjusting the concentrations of corn and soybean meal ingredients, as well as other minor ingredients commonly used in poultry production. Mixing equipment was flushed with ground corn prior to each diet preparation. All diets were prepared using a paddle mixer. The mixer was cleaned between each diet using compressed air and vacuum, mixing equipment was flushed with ground corn between each treatment group, and flush material was retained for disposal.

Diet and water administration: Diets were fed in three feed phases: Starter diet (0-10 days of age), Grower diet (11-24 days of age) and Finisher diet (25-42 days of age). All diets were offered ad libitum, without restriction. Fresh well water (from the research facility deep well) was provided ad libitum.

Feed Formulation Parameters:

| | | | Finisher |
|---|---|---|---|
| Ingredients, % | Starter (d0-11) | Grower (d11-24) | (d24-42) |
| Corn | 58.353 | 65.383 | 69.413 |
| SBM, 47.9% CP | 29.992 | 20.404 | 16.879 |
| Anim & veg fat | 0.05 | 0.05 | 0.050 |
| DL-methionine | 0.515 | 0.4.77 | 0.420 |
| Salt | 0.342 | 0.332 | 0.332 |
| L-Lysine-HCL | 0.367 | 0.439 | 0.400 |
| Limestone | 1.794 | 1.537 | 1.470 |
| Dical Phos | 1.079 | 0.869 | 0.739 |
| Choline CL - 70% | 0.044 | 0.080 | 0.091 |
| TM & Vit Premix | 0.075 | 0.075 | 0.075 |
| Wheat midds | 2.377 | 0.344 | 0.121 |
| Corn DDGs | 5.0 | 10.00 | 10.00 |
| Ronozyme HiPhos | 0.01 | 0.01 | 0.01 |

| Nutrient | Amount | Amount | Amount |
|---|---|---|---|
| Protein, % | 20.3 | 17.5 | 16.0 |
| Ca, % | 0.920 | 0.770 | 0.720 |
| Tphos, % | 0.656 | 0.578 | 0.533 |
| Avphos, % | 0.350 | 0.300 | 0.270 |
| Sodium, % | 0.160 | 0.160 | 0.160 |
| ME kcal/kg | 2900 | 3040 | 3084 |
| Av Lys, % | 1.33 | 1.140 | 1.010 |
| Av M + C, % | 0.99 | 0.880 | 0.790 |
| Choline, g/kg | 1.60 | 1.60 | 1.60 |

Measurement and sampling schedule: On days 0, 10, 24 and 42: Performance; BWG, Fl and FCR (corrected and uncorrected for mortality) Per pen basis. On days 24 and 42: Cecal samples (1 bird/pen), 21 reps/trt; lleal tissue (1 bird/pen), 21 reps/trt; Plasma (1 bird/pen), 21 reps/trt. On day 0 (before bird placement) and day 42: Litter samples (one composite sample per pen), 21 reps/trt (3 in front, 3 in the middle, and 3 in the back).

Results: The test period began on Trial Day 0 (day of hatch of chicks), and chicks were fed a commercial-type feed in pelleted form (crumbles on Days 0-10) until the end of the study. Each treatment contained 21 replicates per treatment randomly assigned and containing 40 male broilers per replicate. Chicks were randomly assigned to treatments on Trial Day 0 (or day of hatch). At 42 days of age, live performance (growth weight gain, mortality and feed conversion) and other criteria were determined.

With respect to daily observations, each pen was closely monitored each day to determine overall health, bird behavior and/or evidence of toxicity, and environmental conditions. Temperature was checked within the growing area employed for this study daily. Temperature program employed for this study was maintaining temperatures of approximately 86+/−5° F. for the first seven (7) days, decreasing approximately 1° F. per day thereafter until a target of approximately 70+/−5° F. was reached, which was maintained throughout the study.

For the entire grow-out period (Days 0-42), body weight gain showed significant improvement over the Control group when broilers were fed diets containing the oligosaccharide preparation Feed conversion for Trial Days 0-42 followed a similar patter as final body weights.

Mortality was considered average for this breed in all groups throughout the growing period to 42 days of age without significant differences. Normal poultry industry mortality is typically <4.5% when birds are grown on litter bedding floors.

Observed data on average body weight, feed conversion ratio (corrected for mortality), mortality in %, and average body weight gain is shown in the subsequent table. Statistical evaluation for each observation is shown in the respective row below.

| Data | Control | Test |
|---|---|---|
| Average body weight in kg, Day 10 | 0.3623 | 0.3684 |
| Statistical evaluation¹ | ^{a} | ^{a} |
| Feed conversion ratio (corrected), Day 0-10 | 1.167 | 1.152 |
| Statistical evaluation¹ | ^{a} | ^{b} |
| Mortality in %, Day 0-10 | 1.19 | 1.31 |
| Statistical evaluation¹ | ^{a} | ^{a} |
| Average body weight gain in kg, Day 0-10 | 0.3184 | 0.3248 |
| Statistical evaluation¹ | ^{a} | ^{a} |
| Average body weight in kg, Day 24 | 1.0484 | 1.0756 |
| Statistical evaluation¹ | ^{a} | ^{b} |
| Feed conversion ratio (corrected), Day 0-24 | 1.413 | 1.379 |
| Statistical evaluation¹ | ^{a} | ^{b} |
| Mortality in %, Day 0-24 | 2.262 | 2.857 |
| Statistical evaluation¹ | ^{a} | ^{a} |
| Average body weight gain in kg, Day 0-24 | 1.0045 | 1.0319 |
| Statistical evaluation¹ | ^{a} | ^{b} |
| Average body weight in kg, Day 42 | 2.5071 | 2.5755 |
| Statistical evaluation¹ | ^{a} | ^{b} |
| Feed conversion ratio (corrected), Day 0-42 | 1.833 | 1.783 |
| Statistical evaluation¹ | ^{a} | ^{b} |
| Mortality in %, Day 0-42 | 3.297 | 4.762 |
| Statistical evaluation¹ | ^{a} | ^{a} |
| Average body weight gain in kg, Day 0-42 | 2.4632 | 2.5318 |
| Statistical evaluation¹ | ^{a} | ^{b} |
| Feed conversion ratio (corrected), Day 10-24 | 1.529 | 1.486 |
| Statistical evaluation¹ | ^{a} | ^{b} |
| Mortality in %, Day 10-24 | 1.09 | 1.579 |
| Statistical evaluation¹ | ^{a} | ^{a} |
| Average body weight gain in kg, Day 10-24 | 0.6861 | 0.7071 |
| Statistical evaluation¹ | ^{a} | ^{b} |
| Feed conversion ratio (corrected), Day 24-42 | 2.136 | 2.075 |
| Statistical evaluation¹ | ^{a} | ^{b} |
| Mortality in %, Day 24-42 | 1.003 | 1.886 |
| Statistical evaluation¹ | ^{a} | ^{a} |
| Average body weight gain in kg, Day 24-42 | 1.4587 | 1.4999 |
| Statistical evaluation¹ | ^{a} | ^{b} |

¹Means within a row without a common superscript are significantly different (P < 0.05) as determined by Least Significant Difference.

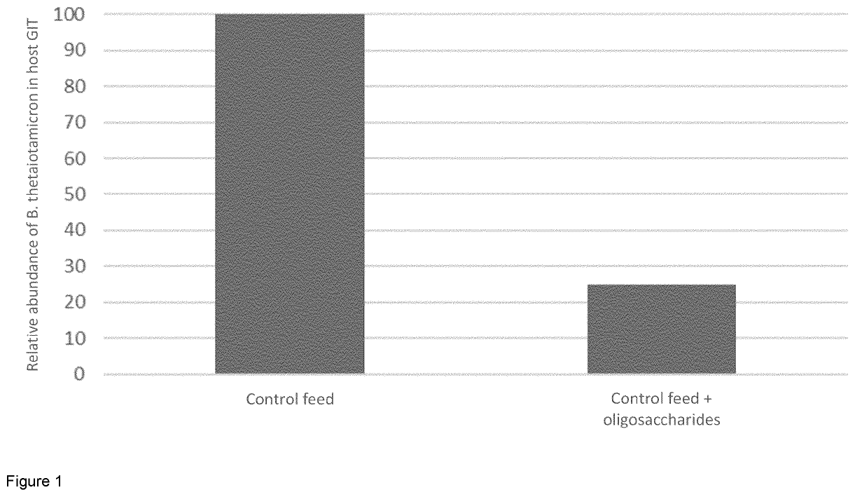

Microbiome analysis: Cecal digesta samples were collected from the control group as well as from the test group (1 bird/pen and 21 replicates/treatment) and treated as described in Example 1. The microbiome of birds that were fed the test group feed, comprising oligosaccharides in form of the oligosaccharide preparation as described above, was found to show an almost 4-fold reduction in Bacteroides thetaiotamicron, FIG. 1. The virulence of enterohaemorrhagic E. coli (EHEC) is reported to be coordinated with the gut commensal Bacteroides thetaiotamicron. Impacting B. thetaiotamicron has subsequent effects on EHEC (Turner et al. Biochem Soc Trans 2019. 47(1): 229-238). The dramatic reduction in B. thetaiotamicron abundance indicates that the synergies between these two organisms have been disrupted, thus effecting a concomitant reduction in E. coli, and in particular a concomitant reduction in exogenous LEE genes and/or non-LEE pathogenic genes of EHEC, EPEC, APEC in the GIT.

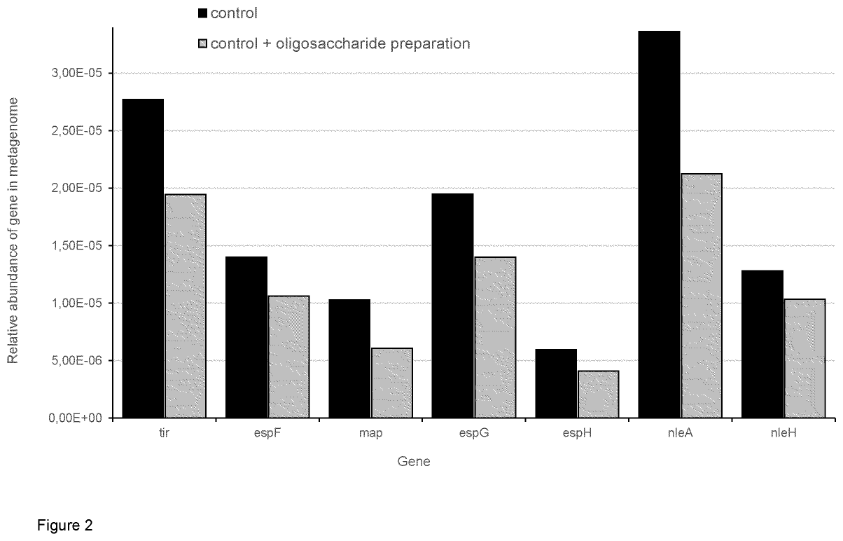

Analysis of abundance of LEE genes and non-LEE pathogenic genes: Samples of GIT content collected at day 14 from the control group as well as from the test group were processed as described in the following. Each GIT sample was extracted for DNA using MoBio Powersoil kit. DNA was then sequenced on an Illumina HiSeq 3000 to produce >2 million random reads of 100 bp representing the DNA of microbes in the GIT. Reads from the sequencing run were aligned using the Burrows-Wheeler alignment algorithm against a reference database of genes that had been previously annotated using KEGG (Kyoto Encyclopedia of Genes and Genomes). It was found, that indeed LEE genes and non-LEE pathogenic genes were downregulated in chicken that were fed with a feed comprising the oligosaccharide preparation described herein. In particular, FIG. 2 shows the reduction in relative abundance of LEE and non-LEE genes in the metagenome of the test group.

1. A method for reducing the population of exogenous locus for enterocyte effacement (LEE) genes and exogenous non-LEE pathogenic genes of Enterohemorrhagic E. coli (EHEC), Enteropathogenic E. coli (EPEC), and Avian Pathogenic E. coli (APEC) in the gastrointestinal tract (GIT) of an animal, comprising feeding said animal with a synthetic oligosaccharide preparation, wherein the population of exogenous LEE genes and non-LEE pathogenic genes is reduced by at least 10% lower than that of a control animal which is fed with the same diet except for said feed additives;
wherein said synthetic oligosaccharide preparation comprises at least n fractions of oligosaccharides each having a distinct degree of polymerization selected from 1 to n (DP1 to DPn fractions), wherein n is an integer greater than or equal to 2; and
wherein each fraction comprises from at least about 0.5% to about 90% (e.g. from 1% to 90%; or e.g. from about 0.5% to about 15%) of anhydro-subunit containing oligosaccharides by relative abundance as determined by mass spectrometry.

2. The method of claim 1, wherein said population of exogenous LEE genes and non-LEE pathogenic genes is measured as % ratio of the combined copy numbers of LEE genes and non-LEE genes detected within the microbiome of said animal vs. the total copy number of genes detected within said microbiome.

3. The method of claim 2, wherein said microbiome is collected from either a fecal sample of the animal or a sample collected within the GIT of the animal.

4. (canceled)

5. (canceled)

6. The method of claim 1, wherein said LEE genes comprise: Tir, Map, EspB, EspF, EspG, EspH, and EspZ.

7. The method of claim 1, wherein said non-LEE pathogenic genes comprise: EspG2, EspJ, EspM1/2, EspT, EspW, Cif, NIeA, NIeB, NIeC, NIeD, NIeE, NIeF, and NIeH.

8. (canceled)

9. The method of claim 1, wherein the concentration of said synthetic oligosaccharide preparation is between 200 and 2000 mg/L of the feed or at least 50 ppm (e.g. at least 50, 70, 100, 150, 200, 300, 400, 500 ppm) of the feed to be given to the group of production animals.

10. (canceled)

11. The method of claim 1, wherein said production animals are: broiler chickens, turkeys, ducks, layers, piglets, grower pigs, finisher pigs, and sows.

12. (canceled)

13. (canceled)

14. (canceled)

15. (canceled)

16. (canceled)

17. (canceled)

18. (canceled)

19. method of reducing the population of E. coli in the gastrointestinal tract (GIT) of an animal, comprising feeding said animal with a synthetic oligosaccharide preparation, wherein the systemic inflammation and/or local inflammation of the animal is reduced by at least 10% lower than that of a control animal which are fed with the same diet except for said feed additives;
wherein said synthetic oligosaccharide preparation comprises at least n fractions of oligosaccharides each having a distinct degree of polymerization selected from 1 to n (DP1 to DPn fractions), wherein n is an integer greater than or equal to 2; and wherein each fraction comprises from at least about 0.5% to about 90% (e.g. from 1% to 90%; or e.g. from about 0.5% to about 15%) of anhydro-subunit containing oligosaccharides by relative abundance as determined by mass spectrometry; and wherein said E. coli is pathogenic E. coli.

20. (canceled)

21. The method of claim 19, wherein the pathogenic E. coli comprises EPEC, EHEC, and APEC.

22. The method of claim 19, wherein said population of E. coli in the GIT of the animal is measured as % of copy number of E. coli marker genes within the microbiome of said animal against the total copy number of bacterial marker genes detected within said microbiome.

23. The method of claim 22, wherein said microbiome is collected from the fecal sample of the animal or a sample collected within the GIT of the animal.

24. (canceled)

25. (canceled)

26. (canceled)

27. The method of claim 19, wherein the concentration of said synthetic oligosaccharide preparation is between 200 and 2000 mg/L of the feed or at least 50 ppm (e.g. at least 50, 70, 100, 150, 200, 300, 400, 500 ppm) of the feed to be given to the group of production animals.

28. method for reducing systemic inflammation and/or local inflammation of an animal caused by E. coli infection, comprising feeding said animal with a synthetic oligosaccharide preparation, wherein the systemic inflammation and/or local inflammation of the animal is reduced by at least 10% lower than that of a control animal which are fed with the same diet except for said feed additives;
wherein said synthetic oligosaccharide preparation comprises at least n fractions of oligosaccharides each having a distinct degree of polymerization selected from 1 to n (DP1 to DPn fractions), wherein n is an integer greater than or equal to 2; and wherein each fraction comprises from at least about 0.5% to about 90% (e.g. from 1% to 90%; or e.g. from about 0.5% to about 15%) of anhydro-subunit containing oligosaccharides by relative abundance as determined by mass spectrometry; and wherein said reduction of inflammation is measured as % ratio of the copy number of LEE and non-LEE genes detected within the microbiome of said animal against the total copy number of genes detected within said microbiome.

29. (canceled)

30. The method of claim 28, wherein said microbiome is collected from the fecal sample of the animal or a sample collected within the GIT of the animal.

31. (canceled)

32. (canceled)

33. (canceled)

34. The method of claim 28, wherein the concentration of said synthetic oligosaccharide preparation is between 200 and 2000 mg/L of the feed or at least 50 ppm (e.g. at least 50, 70, 100, 150, 200, 300, 400, 500 ppm) of the feed to be given to the group of production animals.

35. Use of a synthetic oligosaccharide preparation for
a) reducing the population of exogenous locus for enterocyte effacement (LEE) genes and exogenous non-LEE pathogenic genes of Enterohemorrhagic E. coli (EHEC), Enteropathogenic E. coli (EPEC), and Avian Pathogenic E. coli (APEC) in the gastrointestinal tract (GIT) of an animal, wherein the population of exogenous LEE genes and non-LEE pathogenic genes is reduced by at least 10% lower than that of a control animal which is fed with the same diet except for said feed additives;
b) reducing the population of E. coli in the gastrointestinal tract (GIT) of an animal, wherein the systemic inflammation and/or local inflammation of the animal is reduced by at least 10% lower than that of a control animal which are fed with the same diet except for said feed additives; and/or
c) reducing systemic inflammation and/or local inflammation of an animal caused by E. coli infection, wherein the systemic inflammation and/or local inflammation of the animal is reduced by at least 10% lower than that of a control animal which are fed with the same diet except for said feed additives;
wherein said synthetic oligosaccharide preparation comprises at least n fractions of oligosaccharides each having a distinct degree of polymerization selected from 1 to n (DP1 to DPn fractions), wherein n is an integer greater than or equal to 2; and wherein each fraction comprises from at least about 0.5% to about 90% (e.g. from 1% to 90%; or e.g. from about 0.5% to about 15%) of anhydro-subunit containing oligosaccharides by relative abundance as determined by mass spectrometry.

36. (canceled)
